(19) United States
(12) Patent Application Publication
(10) Pub. No.: US 2014/0235842 A1
(43) Pub. Date: Aug. 21, 2014

(54) CONVENIENT AND EFFICIENT PURIFICATION METHOD FOR CHEMICALLY LABELED NUCLEIC ACIDS

(71) Applicant: SNU R&DB FOUNDATION, Seoul (KR)

(72) Inventors: Seong Keun Kim, Seoul (KR); Young Gyu Kim, Seoul (KR); Ji Hee Hwang, Seoul (KR)

(73) Assignee: SNU R&DB FOUNDATION, Seoul (KR)

(21) Appl. No.: 14/228,570

(22) Filed: Mar. 28, 2014

Related U.S. Application Data
(63) Continuation of application No. PCT/KR2012/007870, filed on Sep. 27, 2012.

(30) Foreign Application Priority Data
Sep. 29, 2011 (KR) 10-2011-0099061
Feb. 8, 2012 (KR) 10-2012-0020649
Sep. 20, 2012 (KR) 10-2012-0104438

(51) Int. Cl. C12Q 1/68

(52) U.S. Cl. CPC C12Q 1/6806
USPC 536/23.1

(57) ABSTRACT

The present invention relates to a method for purifying chemically labeled nucleic acids. More particularly, the present invention relates to a convenient and efficient method for purifying labeled nucleic acids in a fast manner with high efficiency from a mixture of unreacted hydrophobic probes, unreacted nucleic acids and labeled nucleic acids.

In particular, the present invention relates to a convenient method for purifying labeled nucleic acids in a fast manner with high efficiency from a mixture of unreacted hydrophobic chemical probes, unreacted nucleic acids and labeled nucleic acids, comprising the steps of reacting hydrophobic chemical probe and nucleic acids in an aqueous solution to prepare a reacted solution; adding organic solvent to the reacted solution and stirring it to obtain a mixed solution; and centrifuging the mixed solution and removing organic solvent phase.

### REFERENCE TO RELATED APPLICATIONS

This is a continuation of pending International Patent Application PCT/KR2012/007870 filed on Sep. 27, 2012, which designates the United States and claims priority of Korean Patent Application No. 10-2011-0099061 filed on Sep. 29, 2011, Korean Patent Application No. 10-2012-0020649 filed on Feb. 28, 2012, and Korean Patent Application No. 10-2012-0104438 filed on Sep. 20, 2012, the entire contents of which are incorporated herein by reference.

### FIELD OF THE INVENTION

The present invention relates to a method for purifying chemically labeled nucleic acids. More particularly, the present invention relates to a convenient and efficient method for purifying labeled nucleic acids in a fast manner with high efficiency from a mixture of unreacted hydrophobic probes, unreacted nucleic acids and labeled nucleic acids.

In particular, the present invention relates to a convenient method for purifying labeled nucleic acids in a fast manner with high efficiency from a mixture of unreacted hydrophobic chemical probes, unreacted nucleic acids and labeled nucleic acids, comprising the steps of reacting hydrophobic chemical probe and nucleic acids in an aqueous solution to prepare a reacted solution; adding organic solvent to the reacted solution and stirring it to obtain a mixed solution; and centrifuging the mixed solution and removing organic solvent phase.

### BACKGROUND OF THE INVENTION

Chemically labeled nucleic acids have been a subject of major interest in various fields of biological science including molecular biology, cell biology, and molecular diagnostics, etc.

In this regard, various methods for preparing labeled nucleic acids by reacting nucleic acids having nucleophilic functional groups such as primary amine group or thiol group with probes such as fluorescent dye or probe, etc. functionalized with an electrophilic functional group and analyzing such labeled nucleic acids are known [Hermanson, G. T. Bioconjugate techniques; Academic Press, 2008.].

For example, in the case of conjugation mediated by a primary amine group, a fluorescent dye comprising an amine-reactive functional group which can react with primary amine groups, such as ester, isothiocyanate, aldehyde, etc. chemically reacts with the primary amine group introduced in nucleic acids, thus forming labeled nucleic acids, and the nucleic acids labeled as above require the step of being purified from unreacted dye and unreacted nucleic acids.

In this regard, as conventional methods of removing unreacted dyes, methods using ethanol precipitation, size-exclusion chromatography and dialysis are known [Giusti, W. G.; Adriano, T. PCR methods and applications 1993, 2, 223], but these methods mostly have disadvantages such as that they are time consuming, inconvenient and inefficient.

Meanwhile, the technical trend relating to purification methods for labeled nucleic acids is as follows:

JP 2003-511046 A discloses a method for removing unincorporated dye labeled molecules from a mixture comprising a plurality of fluorescent dye labeled polynucleotides and unincorporated fluorescent dye labeled molecules not attached to polynucleotides by contacting the mixture with a plurality of particles that are composed of a cross-linked hydrophilic polymer matrix in which are entrapped hydrophobic porous adsorbent materials to which the unincorporated fluorescent dye labeled molecules can adsorb, so that the unincorporated fluorescent dye labeled molecules pass rapidly through the hydrophilic matrix and become adsorbed onto the hydrophobic material, and the dye-labeled polynucleotides pass through the hydrophilic matrix much more slowly due to their size.

US 20100240103 A1 relates to a method for detecting four or less types of oligonucleotides, and discloses a method for separating oligonucleotides attached to a probe by gel electrophoresis using a fluoroscein derivative as a probe.

An academic journal [William G. Giusti et al, Synthesis and Characterization of 5′-Fluorescent-dye-labeled Oligonucleotides, Genome Research, 1993. 2, pp. 223-227] discloses various methods for attaching a probe comprising a fluorescent chromophore to a modified oligonucleotide synthesized by oligo synthesizer, and selectively purifying only the product by using size-exclusion chromatography, HPLC, or ethanol precipitation.

In general, for size-exclusion chromatography, it takes about 30 minutes ˜1 hour; for dialysis, it takes several hours; for electrophoresis gel extraction, it takes about a day.

Thus, the known methods have problems such as that the separation process is inconvenient and efficiency is low. In particular, they have a problem that the purification time is too long, and the purification technologies for modified nucleic acids developed until now have problems that it is difficult to apply them to processes of large scale.

### SUMMARY OF THE INVENTION

Meanwhile, recently, with regard to labeling nucleic acids, hydrophobic probes with excellent light emitting efficiency are being developed, and such hydrophobic probes have properties greatly different from those of hydrophilic probes which have been conventionally used.

The present inventors paid attention to the case of using such hydrophobic labels in labeling nucleic acids, and completed the present invention for providing a method for purifying labeled nucleic acids in high yield in a simpler and faster manner than the conventional purification technologies. Accordingly, it is a purpose of the present invention to provide a convenient and efficient method for purifying labeled nucleic acids from a mixture comprising unreacted probes and labeled nucleic acids in a fast and simple manner and high yield.

The present inventors invented a convenient and efficient method for purifying labeled nucleic acids from a mixture comprising unreacted probes and labeled nucleic acids using the property that unreacted probes and labeled nucleic acids have different solubility from each other in water and organic solvent due to the hydrophobicity of the probes and the hydrophilicity of the nucleic acids.

In particular, in order to achieve the above purpose, the present invention provides a purification method for chemically labeled nucleic acids as follows:

(1) A purification method for chemically labeled nucleic acids comprising:

(a) reacting hydrophobic chemical probe and nucleic acids in an aqueous solution to prepare a reacted solution;

(b) adding organic solvent into the reacted solution and stirring it to obtain a mixed solution; and

(c) centrifuging the mixed solution and removing organic solvent phase.

(2) The purification method for chemically labeled nucleic acids according to (1), characterized in that the hydrophobic chemical probe has a partition coefficient, represented by the following equation (1), of at least 2:

Partition coefficient=[C_{organic}]/[C_{water}] Equation (1)

wherein [C_{organic}] represents a molarity of chemical probe in organic solvent and [C_{water}] represents a molarity of chemical probe in water.

(3) The purification method for chemically labeled nucleic acids according to (2), characterized in that the hydrophobic chemical probe has the partition coefficient of at least 10.

(4) The purification method for chemically labeled nucleic acids according to (2), characterized in that the hydrophobic chemical probe has the partition coefficient of at least 25.

(5) The purification method for chemically labeled nucleic acids according to (2), characterized in that the hydrophobic chemical probe has the partition coefficient of at least 50.

(6) The purification method for chemically labeled nucleic acids according to any one of (1)˜(5), characterized in that the added organic solvent in the step (a) is an organic solvent saturated with water.

(7) The purification method for chemically labeled nucleic acids according to any one of (1)˜(6), characterized in that the volume of the added organic solvent in the step (b) is at least one time of the volume of the reacted solution of step (a).

(8) The purification method for chemically labeled nucleic acids according to any one of (1)˜(7), characterized by further comprising step (d) and carrying out the step (b) and (c) one or more times.

(9) The purification method for chemically labeled nucleic acids according to any one of (1)˜(8), characterized in that the hydrophobic chemical probe is a hydrophobic fluorophore.

(10) The purification method for chemically labeled nucleic acids according to (9), characterized in that the hydrophobic fluorophore is an amine-reactive fluorophore.

(11) The purification method for chemically labeled nucleic acids according to (10), characterized in that the amine-reactive fluorophore is ATTO 550, ATTO 390 or ATTO 647N.

(12) The purification method for chemically labeled nucleic acids according to any one of (1)˜(11), characterized in that the nucleic acids are oligonucleotide.

(13) The purification method for chemically labeled nucleic acids according to any one of (1)˜(12), characterized in that the organic solvent has a log P value of 0.6 to 4.0.

(14) The purification method for chemically labeled nucleic acids according to (13), characterized in that the organic solvent is alcohol having 3 to 6 carbon atoms, diethyl ether, or chloroform.

The purification method of the present invention allows to purify labeled nucleic acids easily within a short period of time. In particular, according to the present invention, it is possible to purify a plurality of oligonucleotides at the same time, and thus the method can be applied to large scale automated facilities. Also, the present invention makes it possible to supply nucleic acids labeled with probes of various types for various uses in a large amount continuously and quickly with high efficiency.

In particular, according to the present invention, it is possible to purify labeled nucleic acids from unreacted probes in a high yield of at least 90% within a very short period of time of a few minutes. Preferably, labeled nucleic acids can be easily purified from unreacted probes in a high yield of at least 95% or at least 97%.

### BRIEF DESCRIPTION OF THE DRAWINGS

FIG. 1 illustrates a purification method for chemically labeled oligonucleotides.

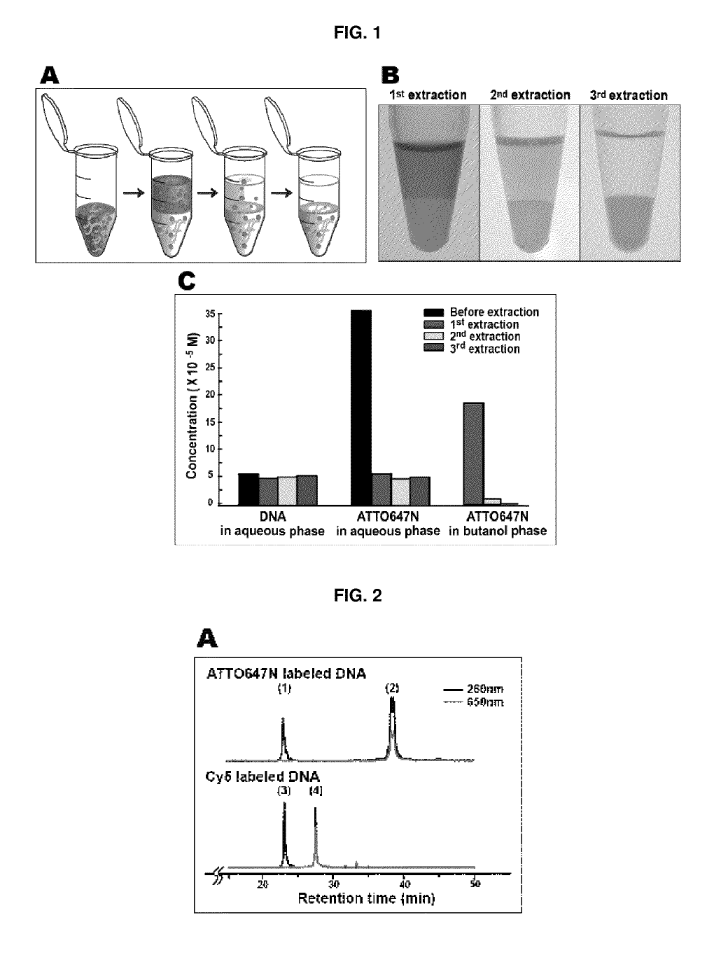

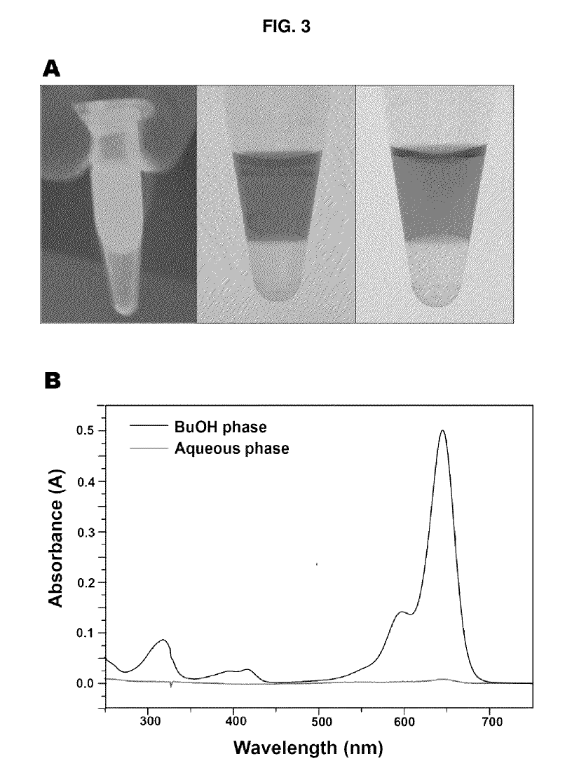

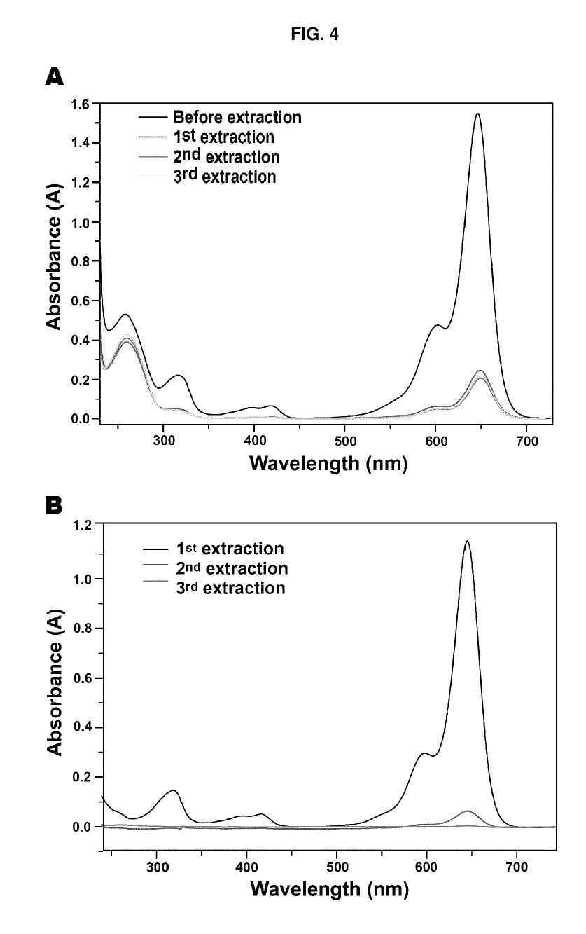

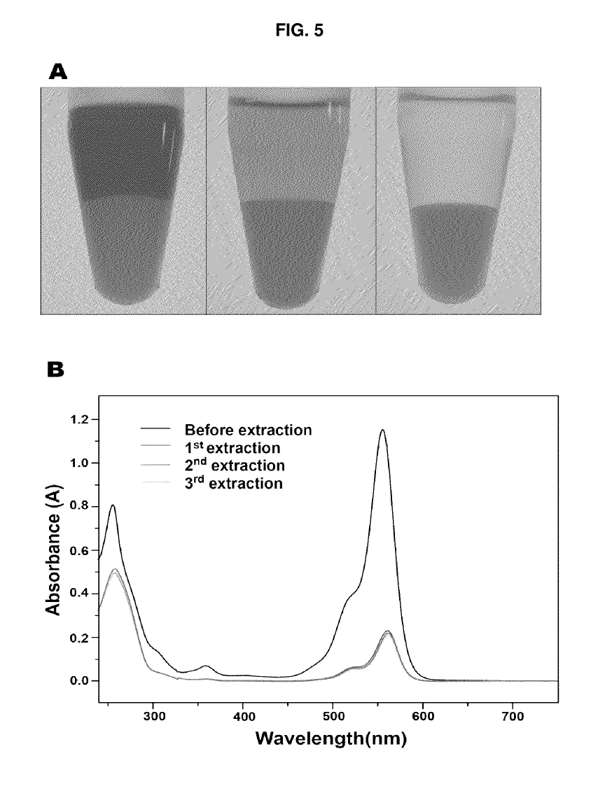

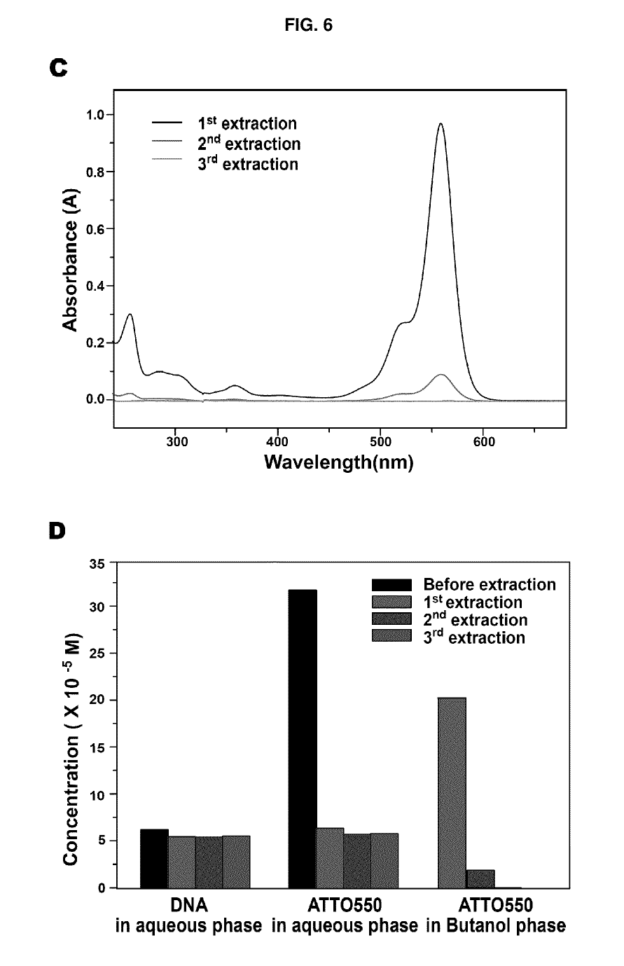

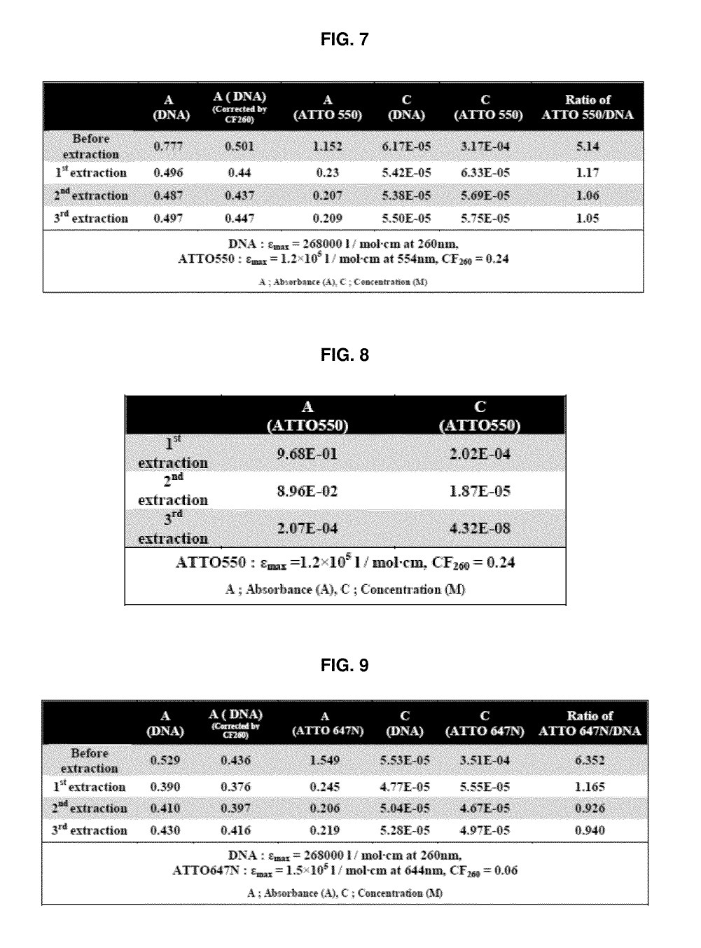

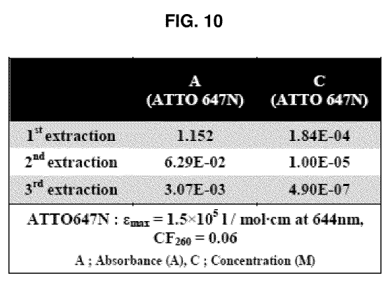

The diagram A in FIG. 1 is a conceptual diagram schematically illustrating a method for removing unreacted dyes by using water-saturated butanol according to the present invention.

The diagram B in FIG. 1 illustrates the extraction of unreacted ATTO 647N dye from the nucleic acids labeling reaction mixture. After the labeling reaction with amine-reactive ATTO, water-saturated butanol is added and the mixed solution is stirred intensely for 10 seconds. Two phases were generated by centrifugation for 10 seconds at 4,000 g, and the organic phase, which is the upper phase was removed. The removal was repeated two more times to completely remove the unreacted dye. The drastic hue change of the upper butanol phase shows that most of the unreacted dye was removed during the first extraction step.

The diagram C in FIG. 1 evaluates the efficiency in removing unreacted dye by extraction. The concentration of DNA in the aqueous phase being constant means that DNA molecules are not separated with butanol phase, and this is also visually evidenced by diagram B showing that the color of the aqueous phase is constant. Also, the concentration of ATTO 647N in the aqueous phase evidences the efficiency of removal after first extraction, and the concentration of ATTO 647N in the butanol phase evidences the color change observed in the butanol phase of the diagram B.

FIG. 2 illustrates the hydrophobic effect of the fluorophore in reverse phase chromatography.

The diagram A in FIG. 2 evaluates the hydrophobicity of ATTO 647N and Cy5 dye. The labeled oligonucleotides was analyzed by High Performance Liquid Chromatography (HPLC) together with μRPC C2/C18 ST 4.6/100 reverse phase column (GE Healthcare). The running conditions was 100 vol % buffer A up to 10 minutes and gradual increase of buffer B up to 50 vol % for 40 minutes. The flow rate was 1 ml/min, and buffers A and B were 0.1 M triethylammonium acetate (TEAA) and 100 vol % acetonitrile (ACN), respectively. The peaks 2 and 4 represent DNA labeled with ATTO 647N and DNA labeled with Cy5, and the peaks 1 and 3 correspond to the unlabeled DNAs in the labeling reaction mixture. The difference in hydrophobicity between Cy5 and ATTO 647N can be confirmed from the fact that the distance between DNA labeled with ATTO 647N and unlabeled DNA is longer than the distance between DNA labeled with Cy5 and unlabeled DNA.

FIG. 3 illustrates the efficiency of the phase extraction method by n-butanol saturated with water.

The photo A in FIG. 3 shows unreacted dyes extracted to the n-butanol phase, which is the upper phase, from the aqueous phase, which is the bottom phase, which include photographs of ATTO 390, 550 and 647N (from left to right in this order). They show that the phase extraction method can be applied to various fluorescent dyes having a certain degree of hydrophobicity.

The diagram B in FIG. 3 is the absorbance spectra of aqueous phase and n-butanol phase after intense mixing. It shows that the unreacted ATTO 647N dyes are partitioned into n-butanol phase.

FIG. 4 shows the efficiency of the phase extracting method using water-saturated n-butanol to separate unreacted ATTO 647N into the n-butanol phase while leaving labeled DNA in the aqueous phase.

The diagram A in FIG. 4 is the absorbance spectra of the aqueous layer during the extraction process. The decrease of the absorbance at 644 nm after the 1st extraction (red) and the constancy of absorbance value after the 2nd and 3rd extractions show that almost all of the unreacted dye is partitioned into the n-butanol phase and only labeled DNA is left in the aqueous phase.

The diagram B in FIG. 4 is the absorbance spectra of n-butanol phase during the process of partitioning unreacted ATTO 647N dyes into n-butanol phase. The decrease of absorbance at 644 nm after the 1st extraction has a difference from the absorbance after the 2nd extraction, thus showing the efficiency of extraction.

FIG. 5 shows the efficiency of the phase extraction method using water-saturated n-butanol to separate unreacted ATTO 550 into n-butanol phase while leaving labeled DNA in aqueous phase.

The photo A in FIG. 5 illustrates extracting ATTO 550 from labeled DNA by using water-saturated n-butanol. Extraction is repeated three times, and the results are shown in the order from left to right. Drastic hue change in the butanol phase (upper phase) shows that most unreacted dyes are removed at the first extraction process.

The diagram B in FIG. 5 is the absorbance spectra of the aqueous phase during the process of separating unreacted ATTO 550 dye into the n-butanol phase.

The diagram C in FIG. 6 is the absorbance spectra of the n-butanol phase during the process of separating unreacted ATTO 550 dye into the n-butanol phase.

The diagram D in FIG. 6 is a graph showing the change of the concentration of ATTO 550 and DNA in both the butanol phase and the aqueous phase.

FIG. 7 is a table showing the absorbance and concentration of each component, ATTO 550 and DNA, in the aqueous phase during extraction.

FIG. 8 is a table showing the absorbance and concentration of each component, ATTO 550 and DNA, in the butanol phase during extraction.

FIG. 9 is a table showing the absorbance and concentration of each component, ATTO 647N and DNA, in the aqueous phase during extraction.

FIG. 10 is a table showing the absorbance and concentration of each component, ATTO 647N and DNA, in the butanol phase during extraction.

### DETAILED DESCRIPTION OF THE INVENTION

Hereinafter, the present invention is explained in more detail.

In general, in order to increase the yield of labeled nucleic acids obtained by the reaction of probes and nucleic acids, an excessive amount of probes are used when compared with the amount of nucleic acids. Thus, unreacted nucleic acids are present in a small amount or hardly exist in the labeling reaction solution, and unreacted probes are present in a large amount.

In case of labeling nucleic acids by using a hydrophobic probe, if an organic solvent is added to a mixture comprising the labeled nucleic acids and unreacted probes, the unreacted probes, which did not react with nucleic acids, are dissolved in the organic solvent during the stirring process because they have a high solubility in organic solvent. If centrifugation is performed after stirring, a small amount of unreacted nucleic acids and a great amount of labeled nucleic acids are present in the aqueous phase and unreacted probes are present in the organic solvent phase; that is, unreacted probes and labeled nucleic acids are separated.

The present invention provides a method for easily purifying labeled nucleic acids selectively from unreacted probes in high yield by using the difference in the solubility in water and organic solvent between nucleic acids which have hydrophilicity and the probes which have hydrophobicity. In one embodiment of the present invention, the method comprises the following steps:

(a) reacting hydrophobic chemical probes and nucleic acids in an aqueous solution to prepare a reacted solution;

(b) adding organic solvent into the reacted solution and stirring it to obtain a mixed solution; and

(c) centrifuging the mixed solution and removing organic solvent phase.

Also, for the case where there is a mixture obtained by reacting hydrophobic chemical probes and nucleic acids, i.e., a mixture comprising unreacted hydrophobic chemical probes and nucleic acids labeled with hydrophobic chemical probes, the present invention provides a method for easily purifying labeled nucleic acids in high yield by adding water and organic solvent to the mixture, stirring it, and centrifuging the mixed solution and removing the organic solvent phase.

In the present invention, the nucleic acids refer to a water soluble polymer comprising sugar, phosphoric acid, and base, and include DNA and RNA. In the present invention, the nucleic acids include oligonucleotides and polynucleotides having more nucleotides than oligonucleotides. In general, oligonucleotide means that the number of the nucleotides is 100 or less or 50 or less.

In the present invention, the nucleic acid is pretreated so that they can be combined with hydrophobic probe, and this can be obtained by introducing a functional group into nucleic acid or purchasing commercially available nucleic acids in which a functional group is already introduced.

As a method for combining nucleic acid with probe, there is a method of preparing labeled nucleic acids by reacting nucleic acids having nucleophilic functional groups such as primary amine group or thiol group with probes such as fluorescent dye or probe, etc. functionalized by electrophilic functional group. The functional group of the nucleic acids and the probe may be connected by a linking group.

The type of functional group present in or newly attached to the nucleic acids is not limited as long as it can be combined with a hydrophobic probe. The functional group can be selected in consideration of the reactivity with the probe. For example, as nucleophilic functional group, primary amine group or thiol group, etc. can be used.

As the method for preparing modified nucleic acids that can be combined with probes by attaching a functional group as above to the nucleic acids, general methods in the technical field of the present invention, in addition to the above exemplified method, may be used.

In the present invention, the probe means a probe used for detecting nucleic acids in the pertinent art, and it refers to a hydrophobic chemical probe. The chemical probes include optically sensed materials that can be sensed by ultraviolet rays, infrared rays, or visible rays, or sensed by the change of refractive index, etc., or electrically sensible materials.

In the present invention, the term hydrophobic means not being readily miscible with water, and this means that the solubility in organic solvent is higher than that in water. In the present invention, preferably, hydrophobic chemical probes of which the partition coefficient as a difference of its solubility between organic solvent and water, which is represented by the following equation 1, is at least 2 can be used.

partition coefficient=[C_{organic}]/[C_{water}] Equation 1:

(wherein [C_{organic}] represents a molarity of chemical probe in organic solvent, and [C_{water}] represents a molarity of chemical probe in water).

Here, partition coefficient means the ratio of the concentrations of a material in each solution, C_{A} and C_{B}, i.e., K=C_{A}/C_{B}, in case where the material is dissolved in liquids A and B which are not miscible with each other, at a certain temperature and under a certain pressure and reaches equilibrium. The partition coefficient can be obtained by measuring the concentration of the chemical probe in organic solvent and the concentration of the chemical probe in water by using a UV/VIS spectrometer.

The partition coefficient is an index showing in which solvent between water and organic solvent the solubility of a material is higher. In the present invention, it shows the correlation between the hydrophobic chemical probe and organic solvent, which is used in purification of labeled nucleic acids. In the present invention, there is no limitation in the type or species of the hydrophobic chemical probe and organic solvent as long as they satisfy the above partition coefficient.

In the present invention, a hydrophobic chemical probe having a partition coefficient for water and organic solvent of at least 2, at least 10, at least 25, at least 50, at least 100, at least 200, or at least 300 is preferably used, and the higher the partition coefficient is, the more preferable the chemical probe is, because as the partition coefficient increases, it becomes easier to separate the labeled nucleic acids and unreacted probes.

As examples of the hydrophobic chemical probe, there are hydrophobic fluorophore, hydrophobic biochemical probe, etc., and they can be used being combined with each other. As examples of the hydrophobic fluorophore, there are fluorescent dyes of ATTO series, etc., and as examples of hydrophobic biochemical probe, there are cholesterol, pyrene, digoxigenin, etc., but they are not limited thereto. Among them, the commercially available hydrophobic fluorophores can be preferably used as the probe of the present invention since it can be easily detected optically.

The hydrophobic fluorophore includes an amine-reactive fluorophore, and in one embodiment of the present invention, it can be used as a hydrophobic probe of the present invention. As examples of amine-reactive fluorophore, there is ATTO 550, ATTO 390 or ATTO 647N, etc., but they are not limited thereto.

The hydrophobic chemical probe of the present invention can be combined with nucleic acids through a functional group that can react with the functional group of the nucleic acid, and the hydrophobic chemical probe already includes or can newly include such functional group, in the structure.

In this regard, there is no limitation in the type of the functional groups which the hydrophobic chemical probe has, and it can be selected in consideration of the reactivity with the functional group introduced in the nucleic acids. For example, in case the functional group of the nucleic acid is a primary amine group, examples of the hydrophobic chemical probe can include functional groups such as ester, isothiocyanate, isocyanate, acyl azide, NHS ester, sulfonyl chloride, aldehyde, etc., and in case the functional group of the nucleic acid is thiol group, examples of the hydrophobic probe can include functional groups such as haloacetyl, alkyl halide derivative, maleimide, aziridine, etc.

The organic solvent used in the present invention is an organic solvent whose layer can be separated from water, and there is no particular limitation in its type as long as it is an organic solvent whose layer can be separated from water.

Thus, in the present invention, an organic solvent having a log P value of at least 0.6 is preferably used, since in case the log P value is less than 0.6, water is not separated from the organic solvent phase. Here, the log P value means the log value of P meaning a partition coefficient of the organic solvent with respect to a mixed solution of the same mol of octanol and water.

Meanwhile, although the upper limit of the log P value of the organic solvent is not limited, in case the log P value is too high, the chemical probe may be more dissolved in water than the organic solvent. A suitable organic solvent is selected by checking whether the hydrophobic chemical probe and organic solvent satisfy the condition that the partition coefficient represented by equation 1 above is at least 2.

The organic solvent that can be preferably used with a general hydrophobic chemical probe is an organic solvent having a log P value within the range of 0.6˜4.0, more preferably, an organic solvent having a log P value within the range of 0.6˜3.5, and most preferably, an organic solvent having a long P value within the range of 0.6˜3.0.

As examples of the organic solvent of which the log P value is within the range of 0.6˜4.0, there are alcohol having 4˜8 carbon atoms such as 1-butanol, isoamyl alcohol, pentanol, etc., ethyl acetate, diethyl ether, diisopropyl ether, butyl acetate, chloroforum, benzene, 1,1,1-trichloroethane, toluene, hexene, etc. In one embodiment, organic solvents may be used in combination with each other.

For example, in case of using amine-reactive fluorophore as a hydrophobic chemical probe, an organic solvent whose log P value is within the range of 0.7˜3.0 can be preferably used. Examples of such organic solvent include alcohol having 4˜8 carbon atoms, diethylether or chloroform, etc. For example, in case of using ATTO 647N as an amine-reactive fluorophore, pentanol can be used as a preferable organic solvent, in case of using ATTO 550, heptanol can be used as a preferable organic solvent, and in case of using ATTO 390, 4-methyl-2-pentanol can be used as a preferable organic solvent.

The reaction of step (a) is performed under ordinary conditions of a labeling reaction of nucleic acids. There is no limitation in the reaction time, and for example, it may be performed at room temperature for 2˜3 hours. Here, preferably, the amount of hydrophobic chemical probe is greater than the mol of nucleic acids. For example, an amount at least 1 time, at least 2 times, at least 5 times, at least 10 times the mol of nucleic acids may be used, and an amount at least 5 times the mol of nucleic acids is preferable.

There is no limitation in the amount of organic solvent added in step (b), but in order to remove the unreacted probe faster and reduce the purification time by reducing the number of purification steps, it is preferable for the volume of the organic solvent to be greater than the volume of the reacted solution of step (a). For example, an organic solvent with a volume at least 2 times, at least 3 times, at least 5 times, or at least 10 times the volume of the reacted solution may be added.

In one embodiment, an organic solvent saturated with water may be used as an organic solvent used in step (b). Here, “organic solvent saturated with water” means an organic solvent which has dissolved as much water as possible and thus does not dissolve water anymore and whose dissolution velocity has reached equilibrium. Here, as the water used to saturate the organic solvent, distilled water is preferably used in order to minimize side effects.

Meanwhile, in case of using an organic solvent that is not saturated with water, during the process of adding an organic solvent and stirring the mixed solution, some of the water of the water phase can be dissolved in the organic solvent phase, and accordingly the amount of water in the water phase may decrease, thus causing enrichment, where the concentration of nucleic acids increases. Also, when organic solvent is added in step (b), an organic solvent in an amount excessive than the amount of the reacted solution of step (a) is added, and accordingly most of the water in the water phase can be dissolved in the organic solvent phase, and thus it may be difficult to partition or separate the water phase comprising labeled nucleic acids and organic solvent phase comprising unreacted probes.

In this regard, in case of using an organic solvent saturated with water, when the organic solvent is added and stirred, the water in the water phase will not be dissolved in the organic solvent phase, and thus it is advantageous in that it does not affect the concentration of the labeled nucleic acids present in the water phase.

There is no limitation in the stirring time in step (b), and even at least several seconds are sufficient. There is no limitation in stirring method, either, which includes methods of simple stirring, shaking, or using instruments such as vortex mixer, etc.

There is no particular limitation in the method of centrifugation in step (c), and the method can be performed by using a centrifugal separator and setting suitable centrifugation conditions. For example, the method may be performed under conditions of 500 g, 1000 g, 2000 g, 4000 g or 6000 g, etc., and may be performed within 10 seconds, 20 seconds, 30 seconds, or 1 minute, and even the time of within 10 seconds is sufficient.

Also, there is no limitation in the method of removing organic solvent phase partitioned by centrifugation; it may be performed by various methods. For example, it may be removed simply by using a pipet.

Among the above steps, if steps (b) and (c) are repeated, the removal yield of unreacted probes increases. Thus, steps (b) and (c) may be performed at least two times, as needed.

Also, the step of removing unreacted nucleic acids can be additionally performed after the step of removing unreacted probe, as needed. In this regard, in case of reacting nucleic acids having a functional group with an excessive amount of probes, the reaction efficiency of the nucleic acids and probes is considerably high. Thus, the step of additionally removing unreacted nucleic acids remaining in the aqueous phase hardly has any meaning. For example, in case of reacting probes with high quality amine-modified oligonucleotide, the reaction efficiency of nucleic acids and probes is considerably high, and thus the additional step of removing unreacted nucleic acids remaining in the aqueous phase hardly has any meaning. However, in order to selectively purify only labeled nucleic acids, after the step of removing unreacted probes, unreacted nucleic acids left in the aqueous phase can be additionally removed, and there is no limitation in the method of removing such unreacted nucleic acids, in the present invention.

As a general method of removing unreacted nucleic acids, chromatography such as reverse phase and ion exchange, or gel electrophoresis can be used. Also, the nucleic acids labeled with hydrophobic probe is partially hydrophobic, which differs from the hydrophilicity of the unreacted nucleic acids, which makes possible simpler purification. For example, labeled nucleic acids and unreacted nucleic acids can be separated from each other through HPLC reverse phase chromatography, and purified using disposable reverse phase column (Polypak, Glen Research, Inc., USA), etc.

By the above method of the present invention, nucleic acids labeled with hydrophobic chemical probe and unreacted probes can be separated from each other in a simple manner in a short period of time of 10 minutes or less, 5 minutes or less, or 3 minutes or less in high yield of at least 90%, at least 95%, or at least 97%.

Hereinafter, the present invention will be explained in more detail by means of the following examples, but the present invention is not limited to the following examples.

### Example 1

A modified oligo nucleotide (30 mer) having an amine group is reacted with fluorescent dye ATTO 647N in a 1.5 ml tube with a reaction ratio of 1:5, and incubated at room temperature for 2˜3 hours.
ATTO 647N dissolved in DMSO (dimethyl sulfoxide): 25 nmole/5.21 μl
DNA dissolved in water: 5 nmole/8.17 μl
0.2M SB buffer (pH 8.5): 20 μl
water: 16.62 μl
entire volume of the reaction solution: 50 μl

2. 200 μl of water-saturated n-butanol is added to the reacted solution, and mixed for about 10 seconds with a vortex mixer, and then centrifuged for about 10 seconds at 4,000 g.

3. The organic phase, which is the upper liquid phase among the solution phases partitioned by step 2, is removed from the solution phases using a pipet.

4. Steps 2˜3 are repeated two more times.

5. The total time spent for performing the above steps 2˜4 is within 3 minutes.

### Example 2

Except for using ATTO 550 instead of ATTO 647N, it is performed in the same manner as example 1.

### Example 3

Except for using ATTO 390 instead of ATTO 647N, it is performed in the same manner as example 1.

The numerical results on the absorbance and concentration of each component and dye in Examples 1 & 2 above are shown in the following Tables 1˜4.

TABLE 1
Nucleic acids labeled with ATTO 647N in an aqueous phase

| | Absorbance | | | | Concentration | Mol of | |
|---|---|---|---|---|---|---|---|
| | of DNA at | Absorbance | Concentration | Mol of | of dye | ATTO | Ratio of |
| | 260 nm | at 649 nm | of DNA | DNA | (M) | 647N | dye/DNA |
| After | 0.436 | 1.55 | 5.53E−05 | 1.11E−08 | 0.000351 | 7.02E−08 | 6.35 |
| reaction | | | | | | | |
| Step 1 | 0.376 | 0.245 | 4.77E−05 | 5.53E−05 | 5.53E−05 | 1.11E−08 | 1.17 |
| Step 2 | 0.397 | 0.206 | 5.04E−05 | 5.53E−05 | 4.67E−05 | 9.34E−09 | 0.926 |
| Step 3 | 0.416 | 0.219 | 5.28E−05 | 5.53E−05 | 4.97E−05 | 9.94E−09 | 0.940 |

TABLE 2
Nucleic acids labeled with ATTO 647N in a butanol phase

| | Absorbance of dye at | | |
|---|---|---|---|
| | 645 nm | Concentration of dye (M) | Mol of dye |
| Step 1 | 1.15 | 0.000184 | 5.52E−08 |
| Step 2 | 0.0629 | 0.0000100 | 3.01E−09 |
| Step 3 | 3.07E−03 | 0.000000490 | 1.47E−10 |

TABLE 3
Nucleic acids labeled with ATTO 550 in an aqueous phase

| | Absorbance | | | | | Mol of | |
|---|---|---|---|---|---|---|---|
| | of DNA at | Absorbance | Concentration | Mol of | Concentration | ATTO | Ratio of |
| | 260 nm | at 649 nm | of DNA | DNA | of dye (M) | 550 | dye/DNA |
| After | 0.504 | 1.41 | 0.0000621 | 1.24E−08 | 0.000388 | 7.76E−08 | 6.26 |
| reaction | | | | | | | |
| Step 1 | 0.395 | 0.207 | 0.0000486 | 9.73E−09 | 0.0000569 | 1.14E−08 | 1.17 |
| Step 2 | 0.406 | 0.191 | 0.0000500 | 1.00E−08 | 0.0000525 | 1.05E−08 | 1.056 |
| Step 3 | 0.401 | 0.187 | 0.0000494 | 9.89E−09 | 0.0000513 | 1.03E−08 | 1.030 |

TABLE 4
Nucleic acids labeled with ATTO 550 in a butanol phase

| | Absorbance of dye at | | |
|---|---|---|---|
| | 559 nm | Concentration of dye (M) | Mol of dye |
| Step 1 | 0.968 | 0.000202 | 5.053E−08 |
| Step 2 | 0.0896 | 0.0000187 | 4.678E−09 |
| Step 3 | 2.07E−04 | 0.0000000432 | 1.08E−11 |

### Reference Example 1

Partition coefficient of ATTO 647N, ATTO 550 and ATTO 390 according to solvent; A_{organic}/A_{water}

TABLE 5

| | 4-methyl- | | | | | | |
|---|---|---|---|---|---|---|---|
| | 2- | | isoamyl | | | | |
| ATTO647N | pentanol | n-butanol | alcohol | pentanol | hexanol | heptanol | octanol |
| A_{organic} | 6.15E−01 | 1.29E+00 | 1.13E+00 | 1.29E+00 | 1.16E+00 | 8.61E−01 | 6.92E−01 |
| A_{water} | 1.83E−01 | 3.20E−02 | 2.24E−02 | 3.30E−03 | 7.10E−03 | 7.11E−02 | 1.41E−01 |
| A_{organic}/A_{water} | 3.36E+00 | 4.02E+01 | 5.06E+01 | 3.90E+02 | 1.64E+02 | 1.21E+01 | 4.92E+00 |

TABLE 6

| | 4-methyl- | | | | | | |
|---|---|---|---|---|---|---|---|
| | 2- | | isoamyl | | | | |
| ATTO550 | pentanol | n-butanol | alcohol | pentanol | hexanol | heptanol | octanol |
| A_{organic} | 4.96E−01 | 6.29E−01 | 5.83E−01 | 5.62E−01 | 5.27E−01 | 5.25E−01 | 5.15E−01 |
| A_{water} | 4.55E−02 | 8.00E−03 | 2.00E−03 | 3.80E−03 | 4.10E−03 | 1.30E−03 | 3.42E−02 |
| A_{organic}/A_{water} | 1.09E+01 | 7.87E+01 | 2.92E+02 | 1.48E+02 | 1.28E+02 | 4.04E+02 | 1.51E+01 |

TABLE 7

| | 4-methyl- | | | | | | |
|---|---|---|---|---|---|---|---|
| | 2- | | isoamyl | | | | |
| ATTO 390 | pentanol | n-butanol | alcohol | pentanol | hexanol | heptanol | octanol |
| A_{organic} | 1.71E+00 | 1.80E+00 | 1.52E+00 | 1.25E+00 | 1.59E+00 | 1.49E+00 | 1.23E+00 |
| A_{water} | 6.00E−04 | 1.50E−02 | 8.60E−03 | 2.31E−02 | 6.90E−03 | 5.00E−03 | 1.00E−03 |
| A_{organic}/A_{water} | 2.85E+03 | 1.20E+02 | 1.77E+02 | 5.39E+01 | 2.31E+02 | 2.98E+02 | 1.23E+03 |

A_{organic}: absorbance in organic solvent
A_{water}: absorbance in water

Using UV/VIS spectrometer, the absorbance of the fluorescent dye dissolved in the corresponding organic solvent and the absorbance of the fluorescent dye dissolved in the water phase are measured. A_{organic} and A_{water} respectively represent the absorbance in the organic solvent and water. The absorbance is proportionate to the concentration, and thus can be converted into the partition coefficient. This is an index representing in which phase between the organic solvent phase and water phase the dye is dissolved better.

ATTO 647N presented the highest partition coefficient for pentanol, and thus showed that it is dissolved better in the pentanol phase than the water phase. Also, it was confirmed that ATTO 550 is best dissolved in heptanol, and ATTO 390 is best dissolved in 4-methyl-2-pentanol.

Through the above results, it can be confirmed that in purifying nucleic acids, an organic solvent having a various range of log P values can be applied to one type of probe, and that the range of the log P value of an organic solvent that can be properly used for a probe having hydrophobicity may vary. Also, it can be anticipated that various organic solvents can be applied to various hydrophobic probes.

### Reference Example 2

Partition coefficient of Cy5 according to solvent

TABLE 8

| Cy5 | 4-methyl-2-pentanol |
|---|---|
| A_{organic} | 1.00E−02 |
| A_{water} | 1.34E+00 |
| A_{organic}/A_{water} | 7.45E−03 |

1. A purification method for chemically labeled nucleic acids comprising:
a) reacting hydrophobic chemical probes and nucleic acids in an aqueous solution to prepare a reacted solution;
b) adding organic solvent into the reacted solution and stirring it to obtain a mixed solution; and
c) centrifuging the mixed solution and removing organic solvent phase.

2. The purification method for chemically labeled nucleic acids according to claim 1, characterized in that the hydrophobic chemical probe has a partition coefficient, represented by the following equation, of at least 2:
Partition coefficient=[Corganic]/[Cwater] Equation (1)
wherein [Corganic] represents a molarity of chemical probe in organic solvent and [Cwater] represents a molarity of chemical probe in water.

3. The purification method for chemically labeled nucleic acids according to claim 1, characterized in that the added organic solvent in the step (b) is an organic solvent saturated with water.

4. The purification method for chemically labeled nucleic acids according to claim 1, characterized by further comprising step (d) and carrying out the step (b) and (c) one or more times.

5. The purification method for chemically labeled nucleic acids according to claim 1, characterized in that the hydrophobic chemical probe is a hydrophobic fluorophore.

6. The purification method for chemically labeled nucleic acids according to claim 5, characterized in that the hydrophobic fluorophore is an amine-reactive fluorophore.

7. The purification method for chemically labeled nucleic acids according to claim 6, characterized in that the amine-reactive fluorophore is ATTO 550, ATTO 390 or ATTO 647N.

8. The purification method for chemically labeled nucleic acids according to claim 1, characterized in that the nucleic acids are oligonucleotides.

9. The purification method for chemically labeled nucleic acids according to claim 1, characterized in that the organic solvent has a log P value of 0.6 to 4.0.

10. The purification method for chemically labeled nucleic acids according to claim 9, characterized in that the solvent is alcohol having 3 to 6 carbon atoms, diethyl ether, or chloroform.
